(19) United States
(12) Patent Application Publication
(10) Pub. No.: US 2007/0260481 A1
(43) Pub. Date: Nov. 8, 2007

(54) Process for evaluating a persons individualized nutritional supplemental needs

(76) Inventor: Richard K. Marshall, Greensburg, PA (US)

Correspondence Address: JAMES RAY & ASSOCIATES, 2640 PITCAIRN ROAD, MONROEVILLE, PA, 15146, US

(21) Appl. No.: 11/429,532

(22) Filed: May 5, 2006

(51) Int. Cl. G06Q 10/00; A61B 5/00

(52) U.S. Cl. 705/2; 600/300

(57) ABSTRACT

A process for evaluating a persons individualized nutritional supplementation needs based on preselected criteria. The process comprises the steps of having a prospective patient obtain a confidential history form and having the prospective patient complete the confidential history form which includes preselected medical information, family history, and preselected lifestyle criteria. This is followed by a step of having the patient authorize review the information that was provided in the previous step by the patient by a preselected assigned doctor and a step of having the patient consent to being treated by the preselected assigned doctor. Ordering laboratory testing of the patient by the preselected assigned doctor is a step in the process which includes providing the patient with a requisition slip for laboratory testing along with directions to a preselected laboratory. This is followed by a step of having at least one of such patient's blood and urine tested at the preselected laboratory and a step of reviewing and evaluating results by the assigned doctor of the tests and a step of providing the patient with a health assessment report by the assigned doctor.

### FIELD OF THE INVENTION

The present invention relates, in general, to dietary supplements, and, more particularly, the present invention relates to a process of evaluating a persons individual medical history along with blood and urine tests as well as other specific tests for determining which supplements such individual should be taking.

### BACKGROUND OF THE INVENTION

We know that over 200 million people are taking nutritional supplements in America today. This is all well and good, but, the real question should be, “Are Americans healthier today than they were ten years ago?”. Cancer used to affect one in ten people. In the next decade it will be one in every two persons. Heart disease is still number two on the list with cancer now becoming number one. So, with all these people taking nutritional supplements in order to become healthier, why are they not healthier?

Quite simply these people do not have an individualized, customized plan for themselves. Many of these people will take whatever the “hot” supplement is for the week, as advertised on television. A large number of these people will stay on this supplement until one bottle is gone and then move on to the next “big” thing in the supplement world. Most of these people do not know what to take, how much to take, when to take it or where to buy it. Further, it is important to note that one of the problems with supplements is that there is no guarantee on the potency or purity of those available across the counter. The most commonly used supplements are those of a first type which are generally the cheapest and are those available at supermarkets and other similar stores, through catalogs or through the internet. These supplements usually contain a lot of fillers, are mass produced and contain the cheapest raw materials. Many times these supplements come in a form that are not able to be utilized by the body. These supplements are usually sold as hard tablets because they are the cheapest to make. Often absorption does not occur and the supplement is excreted from the body, many times still intact.

A second type of supplements are a small step up from the first category. Usually this type of supplement would be available at a health food store or a vitamin store. These supplements are usually more expensive, although most of these supplements are not much better quality than the first type. Thus, the consumer is not always getting what he hopes for when taking across the counter supplements.

It is difficult for an individual to determine whether or not nutritional supplements are necessary and if they are necessary which ones are necessary. To determine this, it is important that laboratory testing be done and it is equally important to have the results of these tests evaluated by a doctor that is trained clinical nutrition in order to evaluate what supplements a person should take. Laboratory results can provide the doctor with the data needed for a personalized nutritional supplement program. Other factors that can affect what supplements are necessary would be, race, gender, age, family history, lifestyle, symptoms/complaints and overall state of health. These are all necessary in order to determine the need on an individual basis.

The doctor trained in clinical nutrition can review the results and determine the nutritional supplements necessary on an individual basis and not have a person take a supplement just because their friend is taking a certain supplement.

Millions of Americans are foolishly spending their money on the current hot supplement or the latest diet trend, but statistics tell us that Americans are getting fatter and more unhealthy by the second. Many of them are wasting their time and money taking supplements they don't need and even taking the wrong supplements.

Further, over 80% of Americans, including children, are taking nutritional supplements without ever having had laboratory tests performed to evaluate which nutritional supplements they should be taking. Properly analyzed by a trained doctor these tests are invaluable for providing an individualized nutritional supplementation plan that will lead a patient down the road to optimum wellness. Thus, it is important to have a nutritional supplement plan and to follow that plan for better health. Following such a plan is not an option, it is essential.

### SUMMARY OF THE INVENTION

In a first aspect the present invention provides a process for evaluating a persons individualized nutritional supplementation needs based on preselected criteria. The process comprises the steps of having a prospective patient obtain a confidential history form and having the prospective patient complete the confidential history form which includes preselected medical information, family history, and preselected lifestyle criteria. This is followed by a step of having the patient authorize review the information that was provided in the previous step by the patient by a preselected assigned doctor and a step of having the patient consent to being treated by the preselected assigned doctor. Ordering laboratory testing of the patient by the preselected assigned doctor is a step in the process which includes providing the patient with a requisition slip for laboratory testing along with directions to a preselected laboratory. This is followed by a step of having at least one of such patient's blood and urine tested at the preselected laboratory and a step of reviewing and evaluating results by the assigned doctor of the at least one of the blood and the urine test and a step of providing the patient with a health assessment report by the assigned doctor.

### OBJECTS OF THE INVENTION

It is, therefore, one of the primary objects of the present invention to provide a process of evaluating a persons individual medical history, including subjective complaints, along with blood and urine tests and other body tests for determining which supplements such individual should be taking.

Another object of the present invention is to provide a process for evaluating which supplements a person should be taking which is convenient for the patient.

Still another object of the present invention is to provide a process for evaluating which supplements a person should be taking which can be done on line through a web site.

Yet another object of the present invention is to provide a process for evaluating which supplements a person should be taking in which the patient can do virtually everything without leaving the home or office.

Another object of the present invention is to provide a process for evaluating which supplements a person should be taking which is monitored by a doctor trained in clinical nutrition.

Still, another object of the present invention is to provide a process for evaluating which supplements a person should be taking which could also involve the doctor making recommendations for changes in a patients lifestyle or dietary changes.

These and various other objects and advantages of this invention will become apparent after a full reading of the following detailed description, particularly, when read in conjunction with the appended claims.

### BRIEF DESCRIPTION OF THE PRESENTLY PREFERRED AND ALTERNATE EMBODIMENTS OF THE INVENTION

The main reason we take nutritional supplements is to repair damage done to the body caused by too much toxin exposure, poor food choices, nutrient depleted diets and stress. A healthy body under ideal circumstances can replace the nutrients it has lost through exercise, toxin exposure, stress, biochemical changes and aging. However, this is not what is really occurring in America. Our bodies are not able to repair and regenerate because we do not provide them with the essential raw materials that are needed for these processes to occur. These raw materials are derived from what we eat and drink. Since most typical diets are filled with salt, sugar, trans-fat, chemicals, preservatives, high calories, low fiber, high saturated fats etc., the body is not getting nearly enough of the healthy nutrients that is needed to restore health and prevent disease. We need to replenish the bodies nutrients so it can maintain a state of optimum wellness.

The present invention provides a process for evaluating a person's individualized nutritional supplementation needs based on preselected criteria. The process comprises the steps of having a prospective patient obtain a confidential history form and having such prospective patient complete the confidential history form which includes preselected medical information, family history, and preselected lifestyle criteria.

Another step is having such prospective patient authorize review of the information that was previously provided by the patient by a preselected assigned doctor and a step of having the prospective patient consent to being treated by the preselected assigned doctor.

There is a step of ordering laboratory testing of the patient by the preselected assigned doctor and providing the patient with a laboratory requisition slip for the testing along with directions to a convenient laboratory and this would be followed by the step of having at least one of the patient's blood and urine tested at such convenient laboratory. This is followed by a step of reviewing and evaluating the test results received from the preselected laboratory by the assigned doctor of the at least one of the blood and urine tests. And, finally there is a step of providing the patient with a health assessment report prepared by the assigned doctor after the doctor has evaluated the results of all of the tests and the patient's confidential history.

The process includes the step of having the prospective patient obtain the confidential history form from a web site, from seeing advertisements on television, radio, in magazines or in newspapers. It is presently preferred that such confidential history form be obtained from a web site and that all communications be done on the internet because of the speed involved in this manner. Communication is done in seconds, whereas, if mailing back and forth is involved it will take at the very least four or five days for a question and a reply to be completed.

Since the web site is the preferred method there is a step of creating a user name and password for the prospective patient so that all of the information will be secure and others cannot access the patient's information.

It should be mentioned that the blood tests ordered by the doctor in one of the process steps may be different for different patients. That is, for example, a patient who suffers from diabetes may in addition to the regular blood test also have to have a fasting blood test performed or a person who is known to have high cholesterol may also have a fasting lipid test performed. This is done so that the doctor can acquire as much information as possible.

An additional step in the process includes having the patient provide samples to the laboratory of one of the patients hair, fingernails, saliva, skin and feces for testing and evaluation.

There is also an additional step of mailing the patient a hair analysis test kit so that the patient can provide a hair sample which can be mailed with the test kit to the preselected laboratory for analysis.

The process further includes an additional step of making nutritional supplementation recommendations by the preselected assigned doctor on at least one of which vitamins, minerals, herbs and nutritional products are needed by the patient and further specifying the exact product, the dosage, and instructions to be used in taking such product, i.e. such as with food or without food, in the morning or at bedtime.

There is, further, a step of instructing the patient on what specialized test should be ordered at a predetermined future time, such future time is generally three months, so that the assigned doctor can evaluate the progress made toward wellness by the patient by using such prescribed product for that period of time. The doctor will then provide the patient with a specific report with the results of the specialized test.

The process also includes the step wherein the doctor will also provide the patient with a maintenance program as a result of the doctor's evaluation of the results of the specialized test for the patient to follow in order to continue to improve and to support changes which have occurred.

This maintenance program will provide the patient with specific information for the patient and a list of supplements and dosages to be taken by the patient for a proscribed period of time. The doctor will also recommend that the patient visit the website every year for an annual evaluation so as to keep up to date on any changes in his/her supplementation needs.

It is a preferred step within the process that such laboratory that is used for the testing is a laboratory that has offices nationwide; however, because of restrictions imposed by various health insurances it is a patients prerogative to use a different laboratory.

It is also within the concept of the process that only pharmaceutical grade supplements will be prescribed for the patients. This is to assure that the patients receive the potency and purity that the doctor has prescribed. Further, the process includes the step of assigning doctors on a geographical basis and, further, assigning at least one preselected doctor for up to one thousand currently active patients.

It is very important that the assigned doctor be trained in the field of clinical nutrition so as to be able to properly evaluate the nutritional requirements of the patient from the results of the blood and urine analysis along with the analysis of the hair as well as the patient's confidential history. Hair is an important screening item since it is the most rapidly growing body component and thus provides much information. Hair is an excretory tissue for essential, nonessential and potentially toxic elements. In general, the amount of an element that is irreversibly incorporated into growing hair is proportional to the level of the element in other body tissues. Therefore, hair elements analysis provides an indirect screening test for physiological excess, deficiency, or mal-distribution of elements in the body. Clinical research has indicated that hair levels of specific elements, particularly potential toxic elements such as cadmium, mercury, lead and arsenic are highly correlated with pathological disorders. For such elements, levels in the hair may be more indicative of body stores than the levels in blood and urine.

The entire process or procedure is set up to be convenient for the patient. The patient has access to nutritional testing through a website. Thus, the patient can spend about 15 minutes filling out the confidential history form on the website and everything, other than the blood work or urine analysis for which the patient must go a convenient laboratory, can be done from the home or office on the internet. This means no more waiting in traffic, or in the doctor's office, missing work for the appointment, using gasoline, or possibly needing babysitters etc.

As stated previously, the procedure is designed for the convenience of the patient. All laboratory requisition forms and test kits are mailed directly to the patient. Any nutritional supplements that are ordered are also shipped directly to the home.

Although it is designed to be convenient as possible for the patient it may be necessary for the assigned doctor to be able to have a physical observation or evaluation of the patient. This may involve the patient being able to transmit photographs of himself/herself by cell phone, Email or direct mail so that the doctor can observe the patient physically. The direct mail may include photographs as a hard copy or photographs on a disc. In some cases it may be necessary for the patient to see the doctor for a physical evaluation.

The question has often been raised, “Why is it necessary to test your blood, urine, and hair?” Through years of research doctors trained in clinical nutrition have been able to see variables in the tests and recommend nutritional supplements to help the body back from a state of sickness to a state of wellness. Since most diseases do not occur overnight, the clinical nutritionist is able to identify and monitor the bodies biochemistry through these tests. The patient is also assured that improvements are being made and that he or she is on the way to wellness. Since clinical nutritionists specialize in nutrition they are trained to recognize subtle irregularities that may exist in these tests. Coupled with the completed history profile form the doctor is able make specific recommendations of what is needed from a nutritional supplementation standpoint. Laboratory testing gives specific information about what is going on inside of a persons body. These tests are critical in determining the state of health and what improvements need to occur.

As was stated previously all nutritional supplements are not created equal. There is no certainty of the purity or potency of most of the supplements available across the counter. The present process deals only with pharmaceutical grade supplements which can only be purchased through doctors. This criteria provides many advantages over the types available across the counter without much of a price difference. These supplements are held to the strictest standards and are made from the best raw materials regardless of cost. They have quality control on every batch of product that is made and there is a chemical assay on every product which guarantees the chemical content, potency and purity. These supplements provide the body with the most bioavailability, absorbability and potency. Thus, it is important to deal with supplements of which there is a certainty of the purity and potency of the supplement so that the individual is assured of receiving what has been prescribed.

Further, because it is possible that some patients may have problems with taking supplements orally or even intravenously, whether they are nutritional supplements or vitamins, some supplements may be administered through the skin by means of patches or by nasal inhalers. The area of transdermal application of nutritional supplements is a growing field and in the future may become a major means of administering supplements to the body. The process further includes a step of providing nutritional supplements for patients which can be administered transdermally or by nasal inhalers.

While a presently preferred embodiment and alternate embodiments of the present invention has been described in detail above, it should be understood that various other adaptations and/or modifications of the invention can be made by those persons who are particularly skilled in the art without departing from either the spirit of the invention or the scope of the appended claims.

I claim:

1. A process for evaluating a persons individualized nutritional supplementation needs based on preselected criteria; said process comprising the steps of:
(a) having a prospective patient obtain a confidential history form;
(b) having said prospective patient complete said confidential history form which includes preselected medical information, family history, and preselected lifestyle criteria which can have an effect on said patient's nutritional needs;
(c) having said patient authorize review of said information provided in step (b) by a preselected assigned doctor;
(d) having said patient consent to being treated by said preselected assigned doctor;
(e) ordering laboratory testing by said preselected assigned doctor, including providing said patient with a requisition slip for said laboratory testing and with directions to a preselected laboratory;
(f) having at least one of said patient's blood and urine tested at said preselected laboratory;
(g) reviewing and evaluating test results received from said preselected laboratory by said preselected assigned doctor of said at least one of said blood and said urine test; and
(h) providing said patient with a health assessment report by said assigned doctor.

2. The process, according to claim 1, wherein said step (a) includes having said prospective patient obtain said confidential history form from one of a web site and from advertising on at least one of television, radio, magazines, and newspapers.

3. The process, according to claim 2, wherein said step (a) includes having said prospective patient obtain said confidential history form by means of said web site.

4. The process, according to claim 3, wherein said process further includes an additional step of creating a user name and password for said prospective patient so as to enable said patient to log on to a secure website.

5. The process, according to claim 1, wherein said process further includes an additional step of having said patient provide samples to said preselected laboratory of at least one of said patients hair, fingernails, skin, feces, and saliva for testing and evaluation.

6. The process, according to claim 5, wherein said process further includes an additional step of mailing said patient a hair analysis test kit so that said patient can provide a hair sample which can be mailed with said test kit to said preselected laboratory for analysis.

7. The process, according to claim 1, wherein said process further includes an additional step of making nutritional supplementation recommendations by said preselected assigned doctor on at least one of which vitamins, minerals, herbs and nutritional products are needed by said patient.

8. The process, according to claim 7, wherein said nutritional products include liquids and solids.

9. The process, according to claim 7, wherein said process further includes an additional step of having said preselected assigned doctor specify an exact product, dosage, and instructions on taking said product.

10. The process, according to claim 9, wherein said process further includes an additional step of instructing said patient what at least one specialized test should be ordered at a predetermined future time so that said assigned doctor can monitor an effectiveness of said treatment while using said prescribed product.

11. The process, according to claim 10, wherein said process includes a plurality of specialized tests.

12. The process, according to claim 10, wherein said process further includes a step of specifying said predetermined future time to be three months.

13. The process, according to claim 10, wherein said process further includes an additional step of providing said patient with a specific report with results of said specialized test.

14. The process, according to claim 10, wherein said process further includes an additional step of providing said patient with a maintenance program prescribed by said preselected assigned doctor as a result of said doctor's evaluation of said results of said specialized tests for said patient to follow in order to continue to improve and support changes which have occurred.

15. The process, according to claim 10, wherein said maintenance program includes providing said patient with specific information for said patient and a list of supplements and dosages to be taken by said patient for a proscribed period of time.

16. The process, according to claim 10, wherein said process further includes an additional step of recommending said patient visit said website every year for an annual evaluation.

17. The process, according to claim 1, wherein said preselected laboratory is a laboratory that has offices nationwide.

18. The process, according to claim 1, wherein said process further includes an additional step of supplying only pharmaceutical grade supplements for said patients.

19. The process, according to claim 1, wherein said process includes assigning said preselected doctors on a geographical basis.

20. The process, according to claim 1, wherein said process includes assigning at least one preselected doctor for up to one thousand currently active patients.

21. The process, according to claim 1, wherein said process further includes an additional step of supplying nutritional supplements which can be administered one of orally, intravenously, and other ways in which supplements can be absorbed by a body.

22. The process, according to claim 1, wherein said other ways include transdermally and by nasal inhalers.

23. The process, according to claim 1, wherein said process further includes an additional step of ordering additional blood tests for patients known to have at least one of diabetes and high cholesterol.

24. The process, according to claim 23, wherein said additional blood tests include fasting glucose and fasting lipids.

25. The process, according to claim 1, wherein said process further includes an additional step of having said patient transmit images of said patient to said preselected assigned doctor by at least one of wireless communication, Email and direct mail.

26. The process, according to claim 25, wherein said wireless communication is by cellular telephone.

27. The process, according to claim 25, wherein said direct mail includes at least one of photographs on hard copy and photographs contained on a disc.
